(19) United States
(12) Patent Application Publication
(10) Pub. No.: US 2003/0044850 A1
(43) Pub. Date: Mar. 6, 2003

(54) Novel chemical library synthesis particles

(76) Inventor: Andrew P. Combs, Kenneth Square, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/229,859

(22) Filed: Aug. 28, 2002

Related U.S. Application Data
(60) Provisional application No. 60/315,575, filed on Aug. 29, 2001.

(51) Int. Cl. G01N 33/53; C12P 21/04; G01N 33/551

(52) U.S. Cl. 435/7.1; 436/524; 428/402

(57) ABSTRACT

Synthesis particles useful for the preparation of chemical libraries and a process of using the same. The particle is of a sufficient size such that it fits into a 384 well or higher density plate and is capable of a loading capacity that allows for numerous assays to be run and then identification by mass spec or NMR of active compounds.

This application claims the benefit of U.S. Provisional Application No. 60/315,575 filed Aug. 29, 2001.

### FIELD OF THE INVENTION

The present invention relates generally to novel synthesis particles useful for the preparation of chemical libraries and processes of using the same.

### BACKGROUND OF THE INVENTION

It is know well know that vast chemical libraries (e.g., combinatorial libraries) can be been formed on particles (e.g., polymeric beads). These libraries are often prepared in such a way that each particle or perhaps a small group of particles contain one discrete molecule or a small number of molecules. The library is then screened for activity. The screening can be performed before or after cleaving the synthesized molecule(s) from the particle. The key to these libraries is being able to identify a molecule after it has been shown to be active.

Two different sized particles have generally been used; (1) small particles (e.g., microparticles) that contain one or just a few compounds within their pores or attached to their surfaces and (2) large particles (e.g., macroparticles) that contain a substantial amount of synthesized compound within their pores or attached to their surfaces.

Small particles tend to range from about 1 to 500 microns in diameter. The limitation of these particles is the ability to have enough material to run numerous assays at low micromolar concentrations (e.g., 10 μM) in a 384 well or higher density plate. Numerous methods have now been developed to identify individual beads and their attached chemical species. These methods include attaching chemical tags, attaching a radio frequency tag (see WO98/46548), encoding particles with high-energy radiation (see WO98/46550), marking the particles with machine readable code (see WO97/15390 and WO99/41006), and forming optical memory devices on or in solid matrices (see U.S. Pat. No. 5,961,923). It can be seen that great and somewhat complicated lengths have been taken in order to identify the compound on a small synthesis particle.

Larger particles can be used to compensate for the lack of material capable of being loaded onto small particles. These particles are generally at least 5 mm in diameter. Sometimes small particle limitations are attempted to be overcome by grafting particles onto a polymeric material that contains numerous beads or placing beads into a container (e.g., a teabag). The substantial increase in size compared with a small particle allows for a much greater amount of material to be loaded onto the particle as compared with small synthesis particles. Chemical syntheses can run followed by cleavage, testing, and identification of active hits. Unfortunately, larger particles are more expensive due to the amount of material in the particle and the amount of material necessary to properly load the particle. Larger particles, also, typically suffer from being less stable and tend to break or facture into smaller particles. In addition, larger particles cannot be used with a standard 384 well or higher density plate.

Consequently, it would be advantageous to discover a synthesis particle that is small enough to be useful in a 384 well or higher density plate, but is large enough to contain enough synthesized material such that numerous low micromolar assays can be run and identification can still be made by standard analytical techniques.

### SUMMARY OF THE INVENTION

Accordingly, one object of the present invention is to provide a novel synthesis particle that fits in a well of a standard 384 well or higher density plate but does not need to be encoded or specially identified to allow for the compounds formed thereon to be structurally identified.

It is another object of the present invention to provide novel process for preparing a chemical library using synthesis particles that can be sorted into the wells of a standard 384 well or higher density plate.

These and other objects, which will become apparent during the following detailed description, have been achieved by the inventors' discovery that the synthesis particles of the present invention can be used in conjunction with standard 384 well or higher density plates, but do not require tedious marking and identification of the individual particles to allow for structurally identification of the synthesized molecules.

### DETAILED DESCRIPTION OF THE INVENTION

Thus, the present invention provides a novel synthesis particle, comprising: a particle that has a diameter greater than 0.5 mm, provided that the particle's diameter does not exceed a diameter sufficient to fit within one well of a standard 384 well plate.

In a preferred embodiment, the particle has a diameter from about 1 mm to 3 mm.

In another preferred embodiment, the synthesis particle has a diameter from about 1 mm to 2 mm.

In another preferred embodiment, the synthesis particle has a diameter from about 1.5 mm to 2 mm.

In another preferred embodiment, the synthesis particle has a loading capacity of from about 0.10 μmoles to 3.0 μmoles.

In another preferred embodiment, the synthesis particle has a loading capacity of from about 0.75 μmoles to 2.0 μmoles.

In another preferred embodiment, the synthesis particle has a loading capacity of from about 0.75 μmoles to 1.5 μmoles.

In another preferred embodiment, the synthesis particle has a loading capacity sufficient to allow from about 1 to 1,000 low micromolar assays to be run.

In another preferred embodiment, about 90% of the particles have a diameter from about 1 mm to 2 mm.

In another preferred embodiment, about 95% of the particles have a diameter from about 1 mm to 2 mm.

In another preferred embodiment, about 90% of the particles have a diameter from about 1.5 mm to 2 mm.

In another preferred embodiment, about 95% of the particles have a diameter from about 1.5 mm to 2 mm.

In another embodiment, the present invention provides a novel process for using a synthesis particle, comprising:

(a) forming a chemical library on a set of synthesis particles, wherein the particles have a diameter of about 1.5 to 3.0 mm;

(b) arraying the particles into an appropriate number of plates;

(c) cleaving the chemical library from the particles; and,

(d) assaying the chemical library.

In another preferred embodiment, about 90% of the particles have a diameter of about 1.5 to 2.0 mm and the plates are 384 well plates.

In another preferred embodiment, about 95% of the particles have a diameter of about 1.5 to 2.0 mm and the plates are 384 well plates.

In another preferred embodiment, the process further comprises:

(e) determining the structures of compounds found active in the assay.

Numerous names have been given to particles useful for chemical library synthesis. Particles, synthesis particles, and beads are used interchangeably herein and are intended to encompass the numerous names given to particles used in the synthesis of chemical libraries. These names include, but are not limited to, insoluble particles, solid support particles, polymer supports, support particles, composite synthesis particles, synthesis particles, synthesis beads, and resin beads.

The shape of the particle includes any that allows the particle to fit in a single well of a standard 384 well or higher density plate (e.g., 1536), depending on the diameter chosen. Preferably, the synthesis particle of the present invention fits in a single well of a 384 well plate. It is to be noted that the particles of the present invention are capable of fitting into plates other than 384 well plates. The well of a 384 well plate is being used herein to define an approximate upper limit of the diameter of the present synthesis particles. Shapes that are contemplated include, but are not limited to, beads (i.e., spheres), pucks, egg-shapes (i.e., elongated spheres), rods, cones, and rings (i.e., doughnut shapes).

Particles, as used herein, is intended to encompass any type of solid article comprised of a material to which can be attached a chemical moiety for the synthesis of a member of a chemical library. The chemical moiety to be attached to the particle can be a linker between the particle and the synthesized chemical compound or a portion of the synthesized chemical compound itself. Preferably, the particles of the present invention do not encapsulate and are not attached to memory devices (e.g., encoded or encodable structures/devices).

The term active site has generally been used to describe the portion of the synthesis particle to which the synthesis of the chemical library begins. Thus, the particles of the present invention comprise active sites or can be modified to comprise active sites. The active sites can be on the surface of the particle or within the pores of the particle. The porosity of the particles of the present invention will depend on the material composition of the particles and the method of manufacture. Both high and low porosity particles are contemplated.

The particles of the present invention can be comprised of one, two, or more layers. Multiple layer particles can be formed in a number of ways including, but not limited to, (a) coating a particle with a second different material or (b) grafting a second material onto the particle. The coating/grafting material can itself contain a group on which the chemical synthesis can begin (i.e., a linker such as an amino-methyl group). Or, the coating/grafting material itself can be modified with a linker or the first portion of the chemical synthesis. The coating/grafting material can be porous or non-porous. Chemical modifications (e.g., aminomethylating the surface and/or pores of a particles) are known to those of skill in the art and are included within the present invention.

The particles are comprised of any material that can be fabricated into a shape useful for the synthesis of a chemical library and is suitably inert to withstand the chemical library synthesis. In other words, the particle is comprised of a material that is not degraded to a point when the compound being synthesized is cleaved from the particle, material released from the particle itself interferes in some way with the syntheses being performed, or the particle changes it shape in such a way that it no longer fits into the 384 well plate (e.g., swelling).

Numerous materials, both natural and synthetic, are known to those of skill in the art to be useful for the preparation of synthesis particles. Examples include, but are not limited to, porous silicates (e.g., sintered glass and controlled pore glass), ceramics, polymers (also called resins) and polymer blends (also called resins) have previously been used. Polymeric materials include, but are not limited to, dextran, celluloses, polysaccharides, polyvinyl pyrrolidine, Teflon® (polytetrafluoroethylene or a derivative thereof), polypropylene, polyesters, polyamides, polyacrylamide, polymethylacrylamide, polyacrylates, polymethacrylates, polystyrenes, and co-polymers, derivatized, and cross-linked versions (e.g., polystyrene cross-linked with divinyl benzene or a derivative thereof) of these materials. A preferred polymeric material is cross-linked polystyrene. Additional particle materials are described by Vaino et al. (J. Comb. Chem. 2000, 2, 579-596), Derek Hudson (J. Comb. Chem. 1999, 1, 403-457), and U.S. Pat. No. 5,961,923. The contents of these publications are incorporated herein by reference. Preferably, the particles are other than glass (e.g., borosilicate materials) or ceramic (e.g., sintered materials).

As noted above, the synthesis particles contain active sites or are modifiable to contain active sites. Co-polymers can be useful materials for the synthesis particles. Preferably, one of the polymers of the co-polymer contains the active site or is modifiable.

The particles of the present invention are intended to be at least 0.5 mm in diameter, provided that the particles fit within one well of a standard 384 well plate. The particles can also fit within higher density plates. Preferably, the particles are of a size that are can be sorted into 384 well plate. The particles of the present invention are also intended to be of a size sufficient to carry an amount of compound that is capable of being tested numerous times and then identified if found active in the test assay. Consequently, the particles of the present invention are intended to be at least 0.5 mm in diameter. Preferably, the particles are at least 1.0 mm in diameter. More preferably, the particles are from about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, to 3.0 mm in diameter. Even more preferably, the particles are about 1.0, 1.1, 1.2, 1.3, 1.4, 1.5, 1.6, 1.7, 1.8, 1.9, to 2.0 mm in diameter. Still more preferably, the particles are about 1.5, 1.6, 1.7, 1.8, 1.9, to 2.0 mm in diameter.

Diameter, as used herein, is intended to represent average diameter (e.g., the sum total of diameters of the beads divided by the number of beads). Average diameters falling within the desired range can be obtained by methods known to those of skill in the art. These can include sieving, manufacturing with stringent tolerances or a combination. Preferably about 90% or more of the particles fall within the stated diameter. More preferably, about 91, 92, 93, 94, 95, 96, 97, 98, 99% or more of the particles fall within the stated diameter. Even more preferably, about 95% or more of the particles fall within the stated diameter. Still more preferably, about 97% or more of the particles fall within the stated diameter.

The process of making the synthesis particles preferably provides particles wherein about 90, 91, 92, 93, 94, 95, 96, 97, 98, to 99% of the particles have diameters falling within 0.2 mm of the desired diameter. More preferably, the process provides particles wherein about 90, 91, 92, 93, 94, 95, 96, 97, 98, to 99% of the particles have diameters falling within 0.1 mm of the desired diameter. Even more preferably, the process provides particles wherein about 90, 91, 92, 93, 94, 95, 96, 97, 98, to 99% of the particles have diameters falling within 0.05 mm of the desired diameter.

The loading capacity of the present particles is dependent upon the porosity of the particles and the chemical bonding sites available. Loading capacity generally refers to the number of active sites (i.e., sites where a growing molecule can be linked) per gram of synthesis particle. In order to have enough material to test and identify, it is preferable that the loading capacity be from about 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, to 3.00 μmoles. More preferably, the loading capacity is from about 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, to 2.00 μmoles. Even more preferably, the loading capacity is from about 0.75, 0.80, 0.85, 0.90, 0.95, 1.00, 1.05, 1.10, 1.15, 1.20, 1.25, 1.30, 1.35, 1.40, 1.45, to 1.50 μmoles.

One of the benefits of the present invention is that more assays can be run than using a small particle, and the structure of the synthesized compound can still be determined by standard techniques (e.g., NMR or LCMS). Preferably, the synthesis particles are loaded with enough material such that after library formation, from about 1, 10, 20, 30, 40, 50, 100, 200, 300, 400, 500, 600, 700, 800, 900, to 1000 assays can be run on the newly prepared compounds. The assays are generally low micromolar concentration of inhibitor assays (e.g., 10 μM assays).

The chemical libraries based on the synthesis particles of the present invention can be prepared by any of the methods known to those of skill in the art (e.g., the combine/mix/divide method and the split synthesis method). The number of reaction steps is dependent upon the desired final products. There can be 1, 2, 3, 4, 5, 6, 7, 8, 9, 10, or more steps. The number of compounds made is dependent upon the desired library. The number of compounds can include from about 1, 10, 10², 10³, 10⁴, 10⁵, 10⁶ or more.

The following order of procedures is preferably used. The chemical library is formed on a set of synthesis particles. The size of the set is determined by the operator and can be based on the number of compounds sought to be prepared. The particles are then arrayed into a plate or plates (e.g., 384 well plates). The number of plates used will be determined by how many particles are in the set and how many particles the operator wishes to place in each well. The compounds thus synthesized are then cleaved from the synthesis particles by methods known to those of ordinary skill in the art. The particles are optionally separated from the cleaved compounds by filtration into another plate. Optionally, the solvents used during cleavage are then removed to leave dried films comprising the synthesized compounds. The compounds are then assayed for activity (i.e., biological activity is determined using a desired assay). Structures of compounds meeting a desired activity level are then determined. Preferably, the structures are determined by NMR or mass spectrometry (e.g., LCMS). It is to be noted that structures of all samples can be determined by LCMS or other known methods.

Numerous modifications and variations of the present invention are possible in light of the above teachings. It is therefore to be understood that within the scope of the appended claims, the invention may be practiced otherwise than as specifically described herein.

What is claimed is:

1. A synthesis particle, comprising: a particle that has a diameter greater than 0.5 mm, provided that the particle's diameter does not exceed a diameter sufficient to fit within one well of a standard 384 well plate.

2. A synthesis particle according to claim 1, wherein the particle has a diameter from about 1 mm to 3 mm.

3. A synthesis particle according to claim 2, wherein the synthesis particle has a diameter from about 1 mm to 2 mm.

4. A synthesis particle according to claim 3, wherein the synthesis particle has a diameter from about 1.5 mm to 2 mm.

5. A synthesis particle according to claim 1, wherein the synthesis particle has a loading capacity of from about 0.10 μmoles to 3.0 μmoles.

6. A synthesis particle according to claim 5, wherein the synthesis particle has a loading capacity of from about 0.75 μmoles to 2.0 μmoles.

7. A synthesis particle according to claim 6, wherein the synthesis particle has a loading capacity of from about 0.75 μmoles to 1.5 μmoles.

8. A synthesis particle according to claim 1, wherein the synthesis particle has a loading capacity sufficient to allow from about 1 to 1,000 low micromolar assays to be run.

9. A synthesis particle according to claim 3, wherein about 90% of the particles have a diameter from about 1 mm to 2 mm.

10. A synthesis particle according to claim 9, wherein about 95% of the particles have a diameter from about 1 mm to 2 mm.

11. A synthesis particle according to claim 4, wherein about 90% of the particles have a diameter from about 1.5 mm to 2 mm.

12. A synthesis particle according to claim 11, wherein about 95% of the particles have a diameter from about 1.5 mm to 2 mm.

13. A process for using a synthesis particle, comprising:
(a) forming a chemical library on a set of synthesis particles, wherein the particles have a diameter of about 1.5 to 3.0 mm;
(b) arraying the particles into an appropriate number of plates;
(c) cleaving the chemical library from the particles; and,
(d) assaying the chemical library.

14. A process according to claim 13, wherein about 90% of the particles have a diameter of about 1.5 to 2.0 mm and the plates are 384 well plates.

15. A process according to claim 13, wherein about 95% of the particles have a diameter of about 1.5 to 2.0 mm and the plates are 384 well plates.

16. A process according to claim 13, further comprising:
(e) determining the structures of compounds found active in the assay.
